(19) United States
(12) Patent Application Publication
(10) Pub. No.: US 2016/0370379 A1
(43) Pub. Date: Dec. 22, 2016

(54) BLOOD-BASED BIOMARKER FOR CANCER

(71) Applicant: J-PHARMA CO., LTD., Yokohama-shi (JP)

(72) Inventors: Hitoshi Endou, Kanagawa (JP); Isao Okayasu, Tokyo (JP); Kiyomi Hana, Kanagawa (JP)

(73) Assignee: J-Pharma Co., Ltd., Kanagawa (JP)

(21) Appl. No.: 14/899,572

(22) PCT Filed: Jun. 9, 2015
(86) PCT No.: PCT/JP2015/066645

§ 371 Date: Dec. 18, 2015

(30) Foreign Application Priority Data
Dec. 22, 2014 (JP) 2014-258445

(51) Int. Cl. G01N 33/68; G06F 19/00

(52) U.S. Cl. CPC G01N 33/6812; G01N 33/6815; G06F 19/3487; G01N 2800/7028; G01N 2800/52

(57) ABSTRACT

A blood-based biomarker for cancer includes a specific amino acid group of at least seven amino acids. The at least seven amino acids include histidine, isoleucine, leucine, methionine, tryptophan, valine, and tyrosine. The specific amino acid group may further include phenylalanine. The cancer is at least one kind of cancer selected from the group consisting of liver cancer, colon cancer, lung cancer, gall bladder cancer, lymph node metastatic cancer, gastric cancer, pancreas cancer, bile duct cancer, breast cancer, malignant mesothelioma, and T-cell leukemia/lymphoma.

### TECHNICAL FIELD

The present invention relates to a blood-based biomarker for cancer.

### BACKGROUND ART

A biomarker for cancer is useful from the viewpoint of prognosis of cancer present in a human body, prediction of efficacy of an anticancer agent, and the like.

For example, a novel polypeptide that can be used as a cancer-specific biomarker and a specific partial peptide thereof are disclosed in Patent Literature 1. Furthermore, a biomarker for cancer using an expression amount of miRNA as an indicator is disclosed in Patent Literature 2. Furthermore, in Patent Literature 3, a biomarker for detecting liver cancer, consisting of a protein which differs in the present or absent, or the amount thereof between a healthy person and a patient with liver cancer is disclosed.

### CITATION LIST

### Patent Literature

Patent Literature 1: JP 2014-14368 A
Patent Literature 2: JP 2014-082953 A
Patent Literature 3: JP 2006-308533 A

### SUMMARY OF THE INVENTION

### Technical Problem

As described above, various biomarkers for cancer have been suggested. However, at the present moment, there is no reliable blood-based biomarker for determining a state of having cancer occurring in each organ or tissue of a human body, that is, a biomarker for determining a cancer-carrying state. Furthermore, like those of Patent Literatures 1 to 3, many biomarkers consist of a specific peptide or a specific nucleic acid, and thus it is not necessarily found to be easily measurable. Therefore, an object of the present invention is to provide a novel blood-based biomarker for cancer enabling reliable determination and simple measurement of a state of having cancer occurring in each organ or tissue of a human body.

### Solution to Problem

The present invention (1) is a blood-based biomarker for cancer, comprising a specific amino acid group of at least seven kinds of amino acids which essentially include histidine, isoleucine, leucine, methionine, tryptophan, valine, and tyrosine.

The present invention (2) is the blood-based biomarker for cancer according to the present invention (1), wherein the specific amino acid group further includes phenylalanine.

The present invention (3) is the blood-based biomarker for cancer according to the present invention (1) or (2), wherein the cancer is at least one kind of cancer selected from the group consisting of liver cancer, colon cancer, lung cancer, gall bladder cancer, lymph node metastatic cancer (for example, porta hepatis and lymph node metastatic cancer), gastric cancer, and pancreas cancer.

The present invention (4) is a method for collecting data for diagnosing cancer in a subject, and the method includes a step of obtaining analysis results of concentration of each amino acid, which is related to the blood-based biomarker according to any one of the present inventions (1) to (3), in blood collected from the subject.

The present invention (5) is the method according to the present invention (4), further including a graphing step for establishing a radar graph of the concentration of each amino acid.

The present invention (6) is a system for collecting data for diagnosing cancer in a subject, and the system includes a means for obtaining analysis results of concentration of each amino acid, which is related to the blood-based biomarker according to any one of the present inventions (1) to (3), in blood collected from the subject.

The present invention (7) is the system according to the present invention (6), further including a graphing means for establishing a radar graph of the concentration of each amino acid.

The present invention (8) is a method for collecting data for evaluating efficacy of an anticancer agent in a subject, and the method includes a step of obtaining analysis results of concentration of each amino acid, which is related to the blood-based biomarker according to any one of the present inventions (1) to (3), in blood collected from the subject.

The present invention (9) is the method according to the present invention (8), further including a graphing step for establishing a radar graph of the concentration of each amino acid.

The present invention (10) is the method according to the present invention (8) or (9), wherein the anticancer agent is an LAT1 inhibitor agent.

The present invention (11) is a system for collecting data for evaluating efficacy of an anticancer agent in a subject, and the system includes a means for obtaining analysis results of concentration of each amino acid, which is related to the blood-based biomarker according to any one of the present inventions (1) to (3), in blood collected from the subject.

The present invention (12) is the system according to the present invention (11), further including a graphing means for establishing a radar graph of the concentration of each amino acid.

The present invention (13) is the system according to the present invention (11) or (12), wherein the anticancer agent is an LAT1 inhibitor agent.

The present invention (14) is a method for collecting data for determining at least one selected from the group consisting of initiating therapeutics with an anticancer agent, confirming a therapeutic effect, and continuation of therapeutics in a subject, and the method includes a step of obtaining analysis results of concentration of each amino acid, which is related to the blood-based biomarker according to any one of the present invention (1) to (3), in blood collected from the subject.

The present invention (15) is the method according to the present invention (14), further including a graphing step for establishing a radar graph of the concentration of each amino acid.

The present invention (16) is the method according to the present invention (14) or (15), wherein the anticancer agent is an LAT1 inhibitor agent.

The present invention (17) is a system for collecting data for determining at least one selected from the group consisting of initiating therapeutics with an anticancer agent, confirming a therapeutic effect, and continuation of therapeutics in a subject, and the system includes a means for obtaining analysis results of concentration of each amino acid, which is related to the blood-based biomarker according to any one of the present inventions (1) to (3), in blood collected from the subject.

The present invention (18) is the system according to the present invention (17), further including a graphing means for establishing a radar graph of the concentration of each amino acid.

The present invention (19) is the system according to the present invention (17) or (18), wherein the anticancer agent is an LAT1 inhibitor agent.

### Advantageous Effects of Invention

According to the present invention, it is possible to provide a novel blood-based biomarker for cancer enabling reliable determination and simple measurement of a state of having cancer occurring in each organ or tissue of a human body. Accordingly, based on the present invention, prognosis of cancer present in a human body can be performed with reliability, efficacy for a cancer patient of a pharmaceutical preparation administered to the cancer patient can be determined with reliability, and also prediction of efficacy of an anticancer agent can be effectively made so that development of new pharmaceuticals can be efficiently performed.

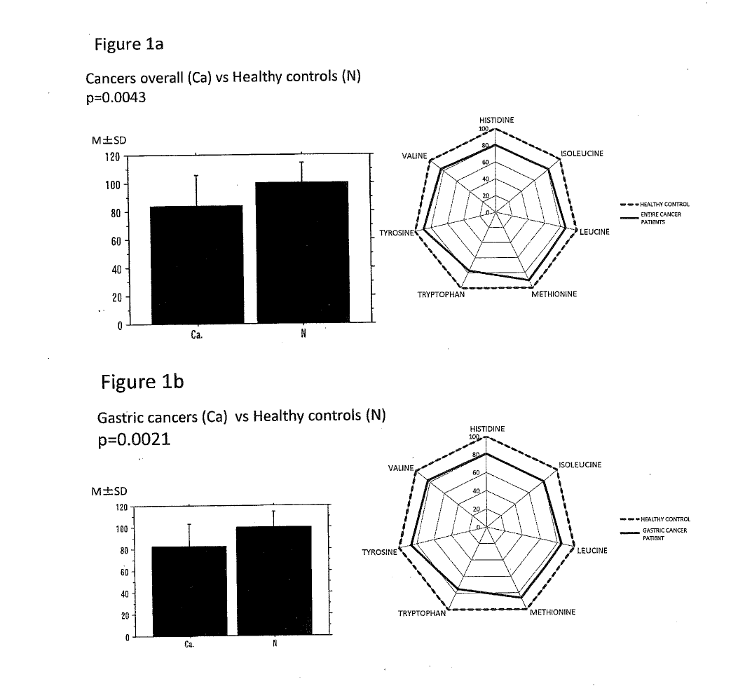

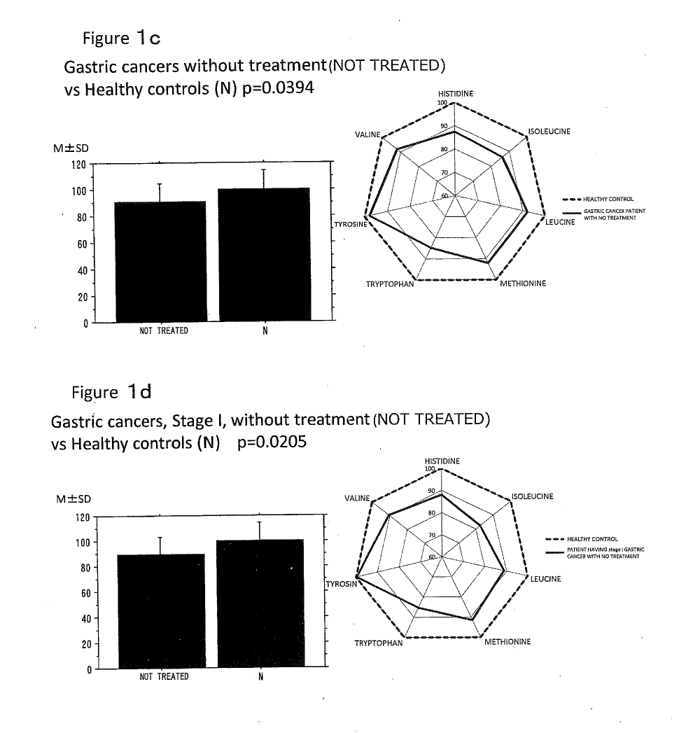

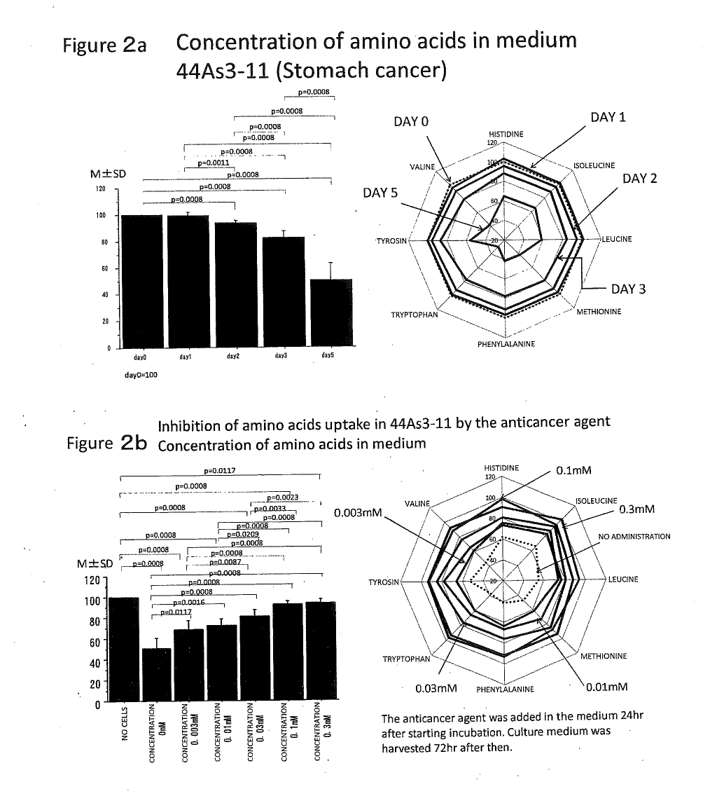

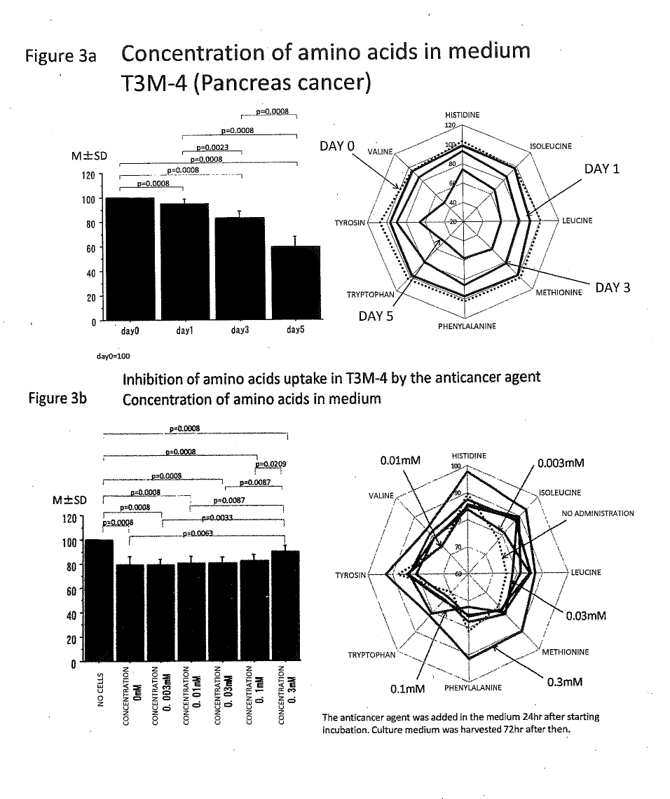

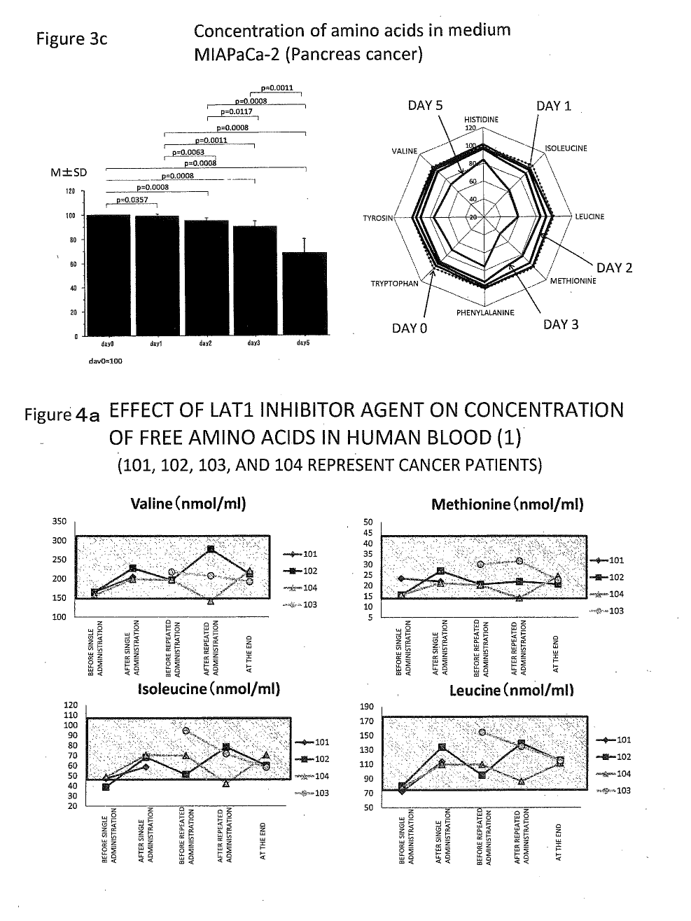

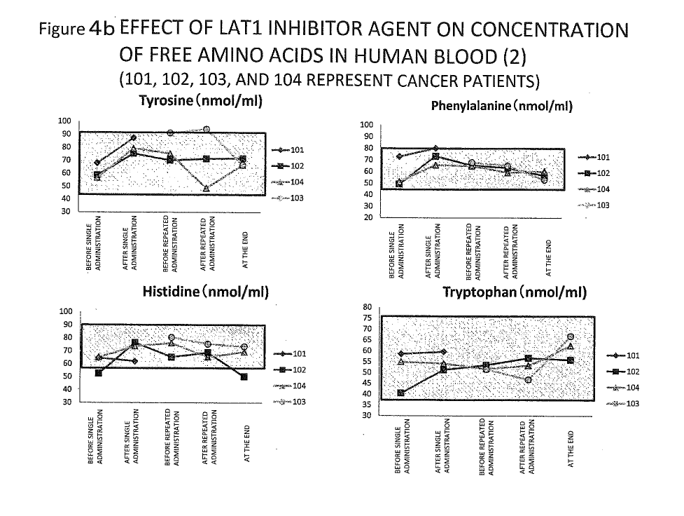

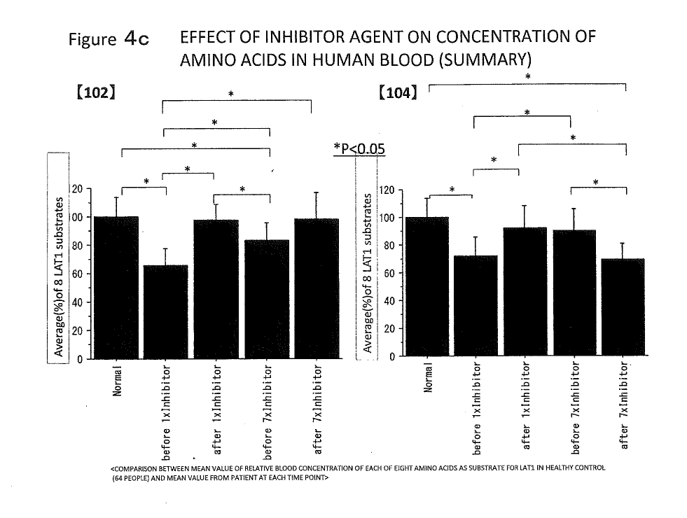

### BRIEF DESCRIPTION OF DRAWINGS

[FIG. 1a] The drawing on the left side illustrates the comparison of mean values of the blood concentration of seven amino acids from a cancer patient tested and a healthy control. The drawing on the right side is a radar graph showing the concentration of each of the seven amino acids.

[FIG. 1b] The drawing on the left side illustrates the comparison of mean values of the blood concentration of seven amino acids from entire gastric cancer patients and a healthy control. The drawing on the right side is a radar graph showing the concentration of each amino acid.

[FIG. 1c] The drawing on the left side illustrates the comparison of mean values of the blood concentration of seven amino acids from a gastric cancer patient who has not been treated and a healthy control. The drawing on the right side is a radar graph showing the concentration of each amino acid.

[FIG. 1d] The drawing on the left side illustrates the comparison of mean values of the blood concentration of seven amino acids from a gastric cancer patient at stage I who has not been treated and a healthy control. The drawing on the right side is a radar graph showing the concentration of each amino acid.

[FIG. 2a] The drawing on the left side illustrates the change in the concentration of eight amino acids in a culture medium of a culture system of gastric cancer cell line 44As3-11. The drawing on the right side is a radar graph showing the concentration of each of the eight amino acids.

[FIG. 2b] The drawing on the left side illustrates the change in the concentration of amino acids in a medium of a culture system of gastric cancer cell line 44As3-11, which LAT1 inhibitor (LAT1 inhibitor agent) has been added. The drawing on the right side is a radar graph showing the concentration of each of the eight amino acids.

[FIG. 3a] The drawing on the left side illustrates the change in the concentration of eight amino acids in a medium of a culture system of pancreas cancer cell line T3M-4. The drawing on the right side is a radar graph showing the concentration of each of the eight amino acids.

[FIG. 3b] The drawing on the left side illustrates the change in the concentration of amino acids in a medium of a culture system of pancreas cancer cell line T3M-4, which LAT1 inhibitor (LAT1 inhibitor agent) has been added. The drawing on the right side is a radar graph showing the concentration of each of the eight amino acids.

[FIG. 3c] The drawing on the left side illustrates the change in the concentration of eight amino acids in a medium of a culture system of pancreas cancer cell line MIAPaCa-2. The drawing on the right side is a radar graph showing the concentration of each of the eight amino acids.

[FIG. 4a] The drawing illustrates the change in concentration of free amino acids of valine, methionine, isoleucine, and leucine in blood serum of a patient at BSC state with administration of LAT1 inhibitor agent.

[FIG. 4b] The drawing illustrates the change in concentration of free amino acids of tyrosine, phenylalanine, histidine, tryptophan in blood serum of a patient at BSC state with administration of LAT1 inhibitor agent.

[FIG. 4c] The drawing illustrates the change in blood concentration of amino acids from a patient who has been administered with LAT1 inhibitor agent, either singly or repeatedly, compared to the blood concentration of amino acids from a healthy control as a reference.

### DESCRIPTION OF EMBODIMENTS

### <<Blood-Based Biomarker for Cancer>>

The blood-based biomarker for cancer according to the present invention is characterized in that the essential components for analysis consist of histidine, isoleucine, leucine, methionine, tryptophan, valine, and tyrosine (hereinbelow, they may be also referred to as “specific amino acids”). Here, in a cancer cell, neutral amino acid transporters (LAT1 and LAT3) for receiving neutral amino acids are specifically present. In this regard, the neutral amino acid transporter receives not only the aforementioned specific amino acids but also neutral amino acids such as arginine, glycine, alanine, serine, threonine, cysteine, asparagine, asparaginic acid, glutamine, glutamic acid, phenylalanine, lysine, proline, or L-DOPA. Thus, in theory, those amino acids can be also expected to function as a biomarker. However, as a result of repeating numerous tests and experiments, it was found that the combination including at least the aforementioned seven specific amino acids is truly effective as a biomarker for cancer, and it constitutes the gist of the present invention.

Here, the amino acids measured as a biomarker are seven kinds of amino acids, that is, histidine, isoleucine, leucine, methionine, tryptophan, valine, and tyrosine. However, as long as they are treated as an essential component, measurement of other amino acids is not excluded. For example, in addition to the aforementioned seven kinds of amino acids, phenylalanine may be taken as an object to be measured. Examples of candidate amino acids as other object to be measured include alanine, arginine, asparagine, asparaginic acid, cysteine, glutamine, glutamic acid, glycine, lysine, phenylalanine, praline, serine, and threonine.

### <<Applications>>

### (Cancer Diagnosis in Subject)

The biomarker of the present invention is effective for cancer diagnosis in a subject. Here, the expression “cancer diagnosis” is a concept which includes not only the determination of a possibility of having cancer in a subject (testee) but also the determination of progress of cancer (progression and/or malignancy). As for the method for diagnosis of cancer in a subject, specific amino acids in blood collected from a subject (histidine, isoleucine, leucine, methionine, tryptophan, valine, and tyrosine) are first quantified as an essential component. Subsequently, as shown in FIG. 1b, for example, concentrations of those specific amino acids are expressed as a radar graph (radar chart). Here, the radar graph described in the present invention is a graph for observing and comparing, at a glance, the size of plural items (at least seven items as specific amino acids). It is desirable that the axis of each item is arranged in a regular polygon form from the center. Furthermore, according to the example of FIG. 1b, it is possible to estimate the possibility of having cancer or severeness of cancer by comparing the “radar graph of a subject” with the “radar graph of an average (healthy control).” Specifically, when a subject has cancer, the overall radar graph of the subject has a shrunken shape compared to the radar graph of an average, depending on the presence or amount of cancer cells. More specifically, when the mean value of the concentration of each amino acid from a healthy control is 100% and the total percentage value of the blood concentration of each amino acid is 700 or less, presence of cancer in a body of the subject is suspected. Meanwhile, the cancer type to which the biomarker of the present invention can be applied is not limited at all, and it is one or more cancer types that are selected from gastric cancer, esophageal cancer, small intestine cancer, colon cancer, cororectal cancer, anal cancer, pancreas cancer, gall bladder cancer, liver cancer, thyroid cancer, adrenal cortex cancer, breast cancer, uterine cancer, cervical cancer, ovary cancer, prostate cancer, testis carcinoma, penis cancer, oral cancer, sialitis cancer, nasopharyngeal cancer, laryngeal cancer, skin cancer, melanoma, soft part sarcoma, bladder cancer, urethra cancer, kidney cancer, mesothelioma, lung cancer, osteosarcoma, Ewing's sarcoma, malignant lymphoma, multiple myeloma, leukemia, brain cancer, and metastatic cancer in various organs or tissues.

Herein, it is desirable that the radar graph is established by using a system equipped with a predetermined program. The system includes a graphing means for establishing a radar graph of the concentration of each amino acid based on the results of analyzing concentration of at least the aforementioned seven specific amino acids in blood, which is collected from a subject. Meanwhile, with the system, the concentration information of the aforementioned seven specific amino acids is obtained, for example, in accordance with input of the results of an analysis (including collecting the information from outside via the Internet or an exclusive line) which has been performed outside. Meanwhile, the system itself may be equipped with a means for analyzing blood, and in such a case, the concentration information of the aforementioned seven specific amino acids can be obtained within the system.

### (Pharmaceutical Efficacy Determination)

The biomarker of the present invention is effective for determination of pharmaceutical efficacy of an anticancer agent. Specifically, as an essential component, specific amino acids (histidine, isoleucine, leucine, methionine, tryptophan, valine, and tyrosine) present in blood, which is collected from a cancer patient who has been administered with a certain anticancer agent, are quantified. Subsequently, as described above in the section of (Cancer diagnosis in subject), the concentration of those specific amino acids is expressed as a radar graph (radar chart). This procedure is repeated over the elapse of time. As a result, determining of whether or not the size of the radar graph of the cancer patient increases over time enables to determine the compatibility of the administered anticancer agent for treating the cancer patient. Accordingly, the method of the present invention is a simple method allowing determination of the appropriateness of continuous administration of an anticancer agent to the cancer patient.

Furthermore, similarly to the section of (Cancer diagnosis in subject) described above, the pharmaceutical efficacy determination is preferably performed by using a system equipped with a predetermined program. The system includes a graphing means for establishing a radar graph of the concentration of each amino acid based on the results of analyzing concentration of at least the aforementioned seven specific amino acids in blood, which is collected from a cancer patient administered with a certain anticancer agent. Meanwhile, similarly to the section of (Cancer diagnosis in subject) described above, with the system, the concentration information of the aforementioned seven specific amino acids is obtained, for example, in accordance with input of the results of an analysis (including collecting the information from outside via the Internet or an exclusive line) which has been performed outside. Meanwhile, similarly to the section of (Cancer diagnosis in subject) described above, the system itself may be equipped with a means for analyzing blood, and in such a case, the concentration information of the aforementioned seven specific amino acids can be obtained within the system. Furthermore, instead of a single analysis after administering an anticancer agent, performing an analysis several times over the elapse of time after administering an anticancer agent is desirable in that the compatibility of the anticancer agent for treating the cancer patient can be appropriately determined. Thus, from this point of view, the system preferably has a recording means for recording the information over the elapse of time from the same person.

### (Development of Anticancer Agent)

The biomarker of the present invention is effective for development of an anticancer agent. Specifically, as an essential component, specific amino acids (histidine, isoleucine, leucine, methionine, tryptophan, valine, and tyrosine) present in blood collected from a cancer patient who has been administered with a certain candidate anticancer agent component are first quantified in a clinical test. Subsequently, as described above in the section of (Cancer diagnosis in subject), the concentration of those specific amino acids is expressed as a radar graph (radar chart). This procedure is repeated over the elapse of time. As a result, determining of whether or not the size of the radar graph of the cancer patient increases over time enable to determine the efficacy of the administered candidate anticancer agent component.

Furthermore, similarly to the section of (Cancer diagnosis in subject) described above, the development of an anticancer agent is preferably performed by using a system equipped with a predetermined program. The system includes a graphing means for establishing a radar graph of the concentration of each amino acid based on the results of analyzing concentration of at least the aforementioned seven specific amino acids in blood, which is collected from a cancer patient administered with a candidate anticancer agent component. Meanwhile, similarly to the section of (Cancer diagnosis in subject) described above, with the system, the concentration information of the aforementioned seven specific amino acids is obtained, for example, in accordance with input of the results of an analysis (including collecting the information from outside via the Internet or an exclusive line) which has been performed outside. Meanwhile, similarly to the section of (Cancer diagnosis in subject) described above, the system itself may be equipped with a means for analyzing blood, and in such a case, the concentration information of the aforementioned seven specific amino acids can be obtained within the system. Furthermore, instead of a single analysis after administering a candidate component, performing an analysis several times over the elapse of time after administering a candidate component is desirable in that the efficacy of the candidate component can be appropriately determined. Thus, from this point of view, the system preferably has a recording means for recording the information over the elapse of time from the same person.

### EXAMPLES

### <<Materials and Methods>>

### 1) Analysis of Patient Blood Serum

Immediately after collecting blood from patients having gastric cancer [100 samples, average age (lowest value-highest value) 66.1 (31 to 89 years old), sex ratio 72:28], pancreas cancer [27, 70.3 (56 to 81) 13:14], or bile duct cancer [6, 70.5 (68 to 75) 5:1] and healthy control with no cancer [12, 50.8 (29 to 73) 11:1], blood serum was isolated and stored in a deep freezer (−80° C.) during a period until determination. The concentration of seven types of amino acids (histidine, isoleucine, leucine, methionine, tryptophan, valine, and tyrosine) was measured by mass analysis, and the measurement result was compared to the value obtained from blood of a healthy control. The concentration of each amino acid was represented as relative value of each case, with the mean value from the healthy control being 100%. The percentage was compared between the cancer patient and healthy control.

### 2) Analysis in Culture System of Cancer Cell Line as Closed Environment, Provided as a Basis of the Invention

Each of the cancer cell lines 44As3-11 (gastric cancer cell line), T3M-4 (pancreas cancer cell line), and MIAPaCa-2 (pancreas cancer cell line) was used. By using a 6 well plate, culture was performed at 37° C. with 5×10⁴ cells in 3 mL (10% FBS, L-Glu+PS in RPMI1640). The culture medium was collected at 0, 24, 48, 72, and 97 hrs and centrifuged at 1,000 rpm. The supernatant (1.0 mL) was then stored in a deep freezer (−80° C.) during a period until determination. Eight kinds of amino acids (including phenylalanine in addition to the aforementioned histidine, isoleucine, leucine, methionine, tryptophan, valine, and tyrosine) were measured by mass analysis. Each culture was performed in triplet and, by measuring the sample, the concentration of each amino acid was represented as mean value±standard deviation, and then compared among the groups. Furthermore, after adding an anticancer agent (O-(5-amino-2-phenylbenzoxazol-7-yl)methyl-3,5-dichloro-L-tyrosine) at each concentration to the culture medium, the property of inhibiting the decrease in amino acid concentration in the culture medium was observed.

3) As for the statistical processing, Mann-Whitney U test was used for comparison between two groups, and those having a risk ratio of 0.05 or less were determined to be a significant difference.

### <<Results>>

### 1) Decrease in Amino Acid Concentration in Blood Serum of Cancer Patient

When compared to the mean value obtained from blood serum of 12 healthy controls, the overall mean value of the concentration of seven amino acids in blood serum of total 131 cancer patients was significantly lower (p=0.0043) (FIG. 1a).
When compared to the mean value obtained from blood serum of 12 healthy controls, the overall mean value of the concentration of seven amino acids in blood serum of total 100 gastric cancer patients was significantly lower (p=0.0021) (FIG. 1b).
When compared to the mean value obtained from blood serum of 12 healthy controls, the overall mean value of the concentration of seven amino acids in blood serum of total 43 gastric cancer patients with no treatment was significantly lower (p=0.0394) (FIG. 1c).
When compared to the mean value obtained from blood serum of 12 healthy controls, the overall mean value of the concentration of seven amino acids in blood serum of total 35 stage I gastric cancer patients with no treatment was significantly lower (p=0.0205) (FIG. 1d) (gastric cancer at early stage is included in the stage I).

### 2) Decrease in Amino Acid Concentration in Culture System of Cancer Cell Lines

Compared to the value at 0 hr of culture, the mean value of the concentration of eight amino acids in the culture medium of the gastric cancer cell line 44As 3-11 exhibited a stepwise decrease as the culture time increases (after 96 hrs, p=0.0008) (FIG. 2a).
Meanwhile, the property of inhibiting amino acid decrease in a pharmaceutical concentration dependent manner was shown in the group in which the LAT1 inhibitor (LAT1 inhibitor agent) is added to the culture medium (FIG. 2b).
Compared to the value at 0 hr of culture, the mean value of the concentration of eight amino acids in the culture medium of the pancreas cancer cell line T3M-4 exhibited a stepwise decrease as the culture time increases (after 96 hrs, p=0.0008) (FIG. 3a).
Meanwhile, the property of inhibiting amino acid decrease in a pharmaceutical concentration dependent manner was shown in the group in which the LAT1 inhibitor (LAT1 inhibitor agent) is added to the culture medium (FIG. 3b).
Compared to the value at 0 hr of culture, the mean value of the concentration of eight amino acids in the culture medium of the pancreas cancer cell line MIAPaCa-2 exhibited a stepwise decrease as the culture time increases (after 96 hrs, p=0.0008) (FIG. 3c).

Meanwhile, the LAT1 inhibitor agent described in the Examples (it may be also expressed as LAT1 inhibitor agent (or, simply inhibitor agent or inhibitor), or the like) indicates O-(5-amino-2-phenylbenzoxazol-7-yl)methyl-3,5-dichloro-L-tyrosine.

<<Analysis of Blood from Cancer Patient at Best Supportive Care (BSC) Stage>>
1) Analysis of Blood Serum from Patient at BSC Stage

A predetermined clinical test (test and experiment) was registered with an administration bureau, and after obtaining an approval of the institutional review board (IRB) of a site management organization, patients were selected and blood serum for analysis was collected, stored, and analyzed. The patient as a subject carried solid tumors for which standard treatments for cancer are ineffective or are intolerable, and they were in a state generally referred to as a BSC stage. The methods for collecting and analyzing blood serum are as described above in the section of <<Materials and Methods>>. The time point for collecting was as follows: (a) before the administration of an inhibitor agent (12 mg/m²/day), (b) 25.5 hours after the start of the initial single administration, (c) before the start of the repeated administration, (d) 25.5 hours after the repeated administration of an inhibitor agent, that is, every day for one week (12 mg/m²/day), and (e) 3 weeks after the end of the repeated administration. The results obtained from those five time points are shown in FIGS. 4a to 4c.

2) The mean value±standard deviation of the relative concentration of each of the eight amino acids from the healthy control (64 people) and each patient was expressed by having a risk ratio of 0.05 or less as a significant difference, while the comparison between the two corresponding groups is made based on the statistical processing method of Student's t test.

### <<Results>>

1) Concentration of Free Amino Acids in Blood Serum from Patient at BSC Stage

For Patient numbers 101 to 104, primary sites and metastatic sites of cancer are as described below.
[Patient number 101] primary; pancreas cancer, metastatic; liver (liver cancer)
[Patient number 102] primary; pancreas cancer, metastatic; liver (liver cancer)
[Patient number 103] primary; colon cancer, metastatic; liver and lung (liver cancer and lung cancer)
[Patient number 104] primary; gall bladder cancer, metastatic; porta hepatis and lymph node metastatic cancer (lymphoma)
Among Patient numbers 101 to 104, 101 was subjected to a single administration only, 103 was subjected to a repeated administration only, and two people of 102 and 104 were subjected to both of a single administration and a repeated administration.
Among the eight amino acids, the results of valine, methionine, isoleucine, and leucine are shown in FIG. 4a and the results of the remaining tyrosine, phenylalanine, histidine, and tryptophan are shown in FIG. 4b. The area surrounded by a box in the drawing means a range in which the blood amino acid concentration is normal.
On the whole, the amino acid value before the single administration was close to the value at the lowest level of normal range, and thus it is believed to be the result showing introduction of blood amino acids to cancer cells, probably mediated by the LAT1.
The concentration of each amino acid after the single administration of an inhibitor agent was higher than the value before the administration. In this regard, it is believed that, as a result of inhibited introduction to cancer that is caused by the LAT1 as believed above, the blood amino acid started to increase, and this is a very important vital reaction.
There can be various changes thereafter, for example, a change in the value before and after the repeated administration. However, compared to the single administration, the biggest difference is that the collecting blood after the administration is performed 3 weeks after the end of the repeated administration. Furthermore, it is believed to be a result of cumulative several factors including a fluctuation in amino acid metabolism in living body among the patients after administering an inhibitor agent (reaction in compliance with the results of newpharmacokinetics).

### 2) Reaction of Inhibitor Agent Having Influence on Blood Amino Acid Concentration

The effect of the inhibitor agent on blood amino acid concentration from subjects 102 and 104, who have been subjected to a single administration and also a repeated administration, is shown in FIG. 4c. When the mean value of the blood concentration of each amino acid from the healthy control (64 people) is 100, the deviation (standard deviation) of each person was shown as normal in the left column of each graph. The mean value±standard deviation of the relative concentration of each of the eight amino acids from each patient was shown with each blood-collecting time point and compared in the four columns at right side. With regard to those two examples, there are two important aspects; firstly, there is a significant difference between the normal and the case before the single administration (before 1× inhibitor), and secondly, there is also a significant difference between cases before and after the single administration.
Like the patient at BSC stage of this test, the effect of the LAT1 on the blood amino acid concentration is high in progressive cancer. Therefore, the eight amino acids have decreased in the blood. Thus, it gives an indication for determining of whether an inhibitor agent is applied to each patient or not. The reaction, due to which the lowered values of eight amino acids increase as a result of administering an inhibitor agent, indicates that the function of the LAT1 has responded to the inhibitor agent.
Furthermore, even for determination of continuation of an inhibitor agent therapeutics, the change in blood amino acid concentration can play a role of a biomarker for cancer, in conjunction with clinical opinion or test opinion, in particular, a change in tumor size.

As described above, according to the present invention, it is possible to provide a simple method in which total concentration value (or, mean value) of specific amino acids from a healthy control is compared with the total concentration value (or, mean value) of specific amino acids from a subject by using seven kinds (or eight kinds) of specific amino acids as a blood biomarker to determine whether or not a state of having cancer occurring in each organ or tissue is seen. Furthermore, the present invention can be applied to cancer therapeutics of a cancer patient, in particular, a system or a method for enabling the feasibility of a personalized medical treatment (system or method for personalized medical treatment) by determining suitability of neutral amino acid transporter or LAT1 selective inhibitor (LAT1 inhibitor agent), or a system or a method (system or method for determination) for determining initiation of a treatment with an inhibitor agent, confirmation of a therapeutic effect, or determining treatment continuance.

1. A blood-based biomarker for cancer, comprising a specific amino acid group of at least seven amino acids, wherein the at least seven amino acids include histidine, isoleucine, leucine, methionine, tryptophan, valine, and tyrosine.

2. The blood-based biomarker for cancer according to claim 1, wherein the specific amino acid group further includes phenylalanine.

3. The blood-based biomarker for cancer according to claim 1, wherein the cancer is at least one kind of cancer selected from the group consisting of liver cancer, colon cancer, lung cancer, gall bladder cancer, lymph node metastatic cancer, gastric cancer, pancreas cancer, bile duct cancer, breast cancer, malignant mesothelioma, and T-cell leukemia/lymphoma.

4. A method for collecting data for diagnosing cancer in a subject, the method comprising obtaining analysis results of concentration of each amino acid, which is related to the blood-based biomarker according to claim 1, in blood collected from the subject.

5. The method according to claim 4, further comprising graphing a radar graph of the concentration of each amino acid.

6. A system for collecting data for diagnosing cancer in a subject, the system comprising an analyzer for obtaining analysis results of concentration of each amino acid, which is related to the blood-based biomarker according to claim 1, in blood collected from the subject.

7. The system according to claim 6, further comprising a graphing means for establishing a radar graph of the concentration of each amino acid.

8. A method for collecting data for evaluating efficacy of an anticancer agent in a subject, the method comprising obtaining analysis results of concentration of each amino acid, which is related to the blood-based biomarker according to claim 1, in blood collected from the subject.

9. The method according to claim 8, further comprising graphing a radar graph of the concentration of each amino acid.

10. The method according to claim 8, wherein the anticancer agent is an LAT1 inhibitor agent.

11. A system for collecting data for evaluating efficacy of an anticancer agent in a subject, the system comprising an analyzer for obtaining analysis results of concentration of each amino acid, which is related to the blood-based biomarker according to claim 1, in blood collected from the subject.

12. The system according to claim 11, further comprising a graphing means for establishing a radar graph of the concentration of each amino acid.

13. The system according to claim 11, wherein the anticancer agent is an LAT1 inhibitor agent.

14. A method for collecting data for determining at least one selected from the group consisting of initiating therapeutics with an anticancer agent, confirming a therapeutic effect, and continuation of therapeutics in a subject, the method comprising obtaining analysis results of concentration of each amino acid, which is related to the blood-based biomarker according to claim 1, in blood collected from the subject.

15. The method according to claim 14, further comprising graphing a radar graph of the concentration of each amino acid.

16. The method according to claim 14, wherein the anticancer agent is an LAT1 inhibitor agent.

17. A system for collecting data for determining at least one selected from the group consisting of initiating therapeutics with an anticancer agent, confirming a therapeutic effect, and continuation of therapeutics in a subject, the system comprising an analyzer for obtaining analysis results of concentration of each amino acid, which is related to the blood-based biomarker according to claim 1, in blood collected from the subject.

18. The system according to claim 17, further comprising a graphing means for establishing a radar graph of the concentration of each amino acid.

19. The system according to claim 17, wherein the anticancer agent is an LAT1 inhibitor agent.
